(19) United States
(12) Patent Application Publication
(10) Pub. No.: US 2018/0008563 A1
(43) Pub. Date: Jan. 11, 2018

(54) PHARMACEUTICAL COMPOSITION FOR PREVENTING AND/OR TREATING DISEASE CAUSED BY CORONAVIRUS AND/OR ROTAVIRUS

(71) Applicants: GENIFARM LABORATORIES INC, Guangdong (CN); GUANG ZHOU YUAN TU BIOLOGICAL ANG CHEMICAL TECHNOLOGY CO., LTD., Guangdong (CN)

(72) Inventors: Yongdong WANG, Guangdong (CN); Jiyuan CAO, Guangdong (CN); Shifa ZHU, Guangdong (CN); Wen CHENG, Guangdong (CN); Zhipeng HUANG, Guangdong (CN)

(73) Assignees: GENIFARM LABORATORIES INC, Guangdong (CN); GUANG ZHOU YUAN TU BIOLOGICAL ANG CHEMICAL TECHNOL OGY CO., LTD, Guangdong (CN)

(21) Appl. No.: 15/543,373

(22) PCT Filed: Jan. 17, 2015
(86) PCT No.: PCT/CN2015/070954

§ 371 Date: Jul. 13, 2017

(51) Int. Cl. A61K 31/185; A61K 31/593; A61K 31/525; A61K 31/519; A61K 31/51; A61K 31/4415; A61K 31/07; A61K 31/4188; A61K 31/375; A61K 31/355; A61K 31/191; A61K 31/122; A61K 31/714; A61K 31/455

(52) U.S. Cl. CPC A61K 31/185; A61K 31/714; A61K 31/593; A61K 31/525; A61K 31/519; A61K 31/51; A61K 31/455; A61K 31/07; A61K 31/4188; A61K 31/375; A61K 31/355; A61K 31/191; A61K 31/122; A61K 31/4415

(57) ABSTRACT

The present invention provides a pharmaceutical composition for prevention and/or treatment of diseases induced by viruses of genus coronavirus and/or genus rotavirus. The pharmaceutical composition includes the following components by weight percent: 0.5-100% of taurine, 0-85% of a vitamin complex, and 0-15% of a flavoring agent.

### CROSS REFERENCE TO RELATED APPLICATION

This application is a national stage entry of PCT/CN2015/070954 filed Jan. 17, 2015, under the International Convention.

### TECHNICAL FIELD OF THE INVENTION

The present invention relates to a technical field of disease prevention and disease treatment, and in particular, relates to a pharmaceutical composition for prevention and/or treatment of diseases induced by viruses of genus coronavirus and/or genus rotavirus.

### BACKGROUND OF THE INVENTION

Viruses of genus coronavirus and genus rotavirus may induce various diseases of livestock, and particularly porcine viral diarrhea induced by a porcine transmissible gastroenteritis virus, a porcine epidemic diarrhea virus and/or a porcine rotavirus and the like has become one of the severest diseases threatening pig production.

Until now, there is no effective prevention or treatment method available for the porcine viral diarrhea. The reasons lie in that, in one aspect, effective and reliable vaccine has not been successfully developed. In recent years, an isolated porcine epidemic diarrhea virus strain has a gene homology of about 86-98% with CV777, which indicates great difficulty of vaccine research and development. In fact, a commercially available PED vaccine or a PED/TGE bivalent vaccine and a PED/TGE/PoRV trivalent vaccine under research and development that have been approved in China, all do not achieve an ideal protection rate on the viral diarrhea. The PED vaccine approved by United States in 2014 is also subject to such problem. Currently, a return feeding technique is widely applied to treat such disease worldwide, especially in the United States, which is also a helpless choice. First, the return feeding needs epidemic materials, in other words, materials for the return feeding may be obtained only in the case of occurrence of the disease, and once the disease occurs, the loss is inevitable. Second, the effect of the return feeding is not definite, and the disease may recur two or three months later. Third, the return feeding may cause biosafety risks, in other words, outbreaks of other diseases may be introduced to swine herds and would lead to an even greater loss.

In another aspect, the conventional drugs for prevention or treatment of the diseases do not achieve satisfactory effect. For example, interferons, immunoglobulin of yolk, thymosin peptide and the like do not achieve an ideal effect on the porcine epidemic diarrhea. Research by Kim et al. suggests that the antiviral drug, Ribavirin, is capable of inhibiting replication of PEDV (Virus Research (2013), 171(1), 44-53). However, using human antiviral drugs for food animals was prohibited by regulations and laws all around the world, leading to that use of the antiviral drugs such as Ribavirin, amantadine and the like is prohibited in prevention and treatment of the porcine epidemic diarrhea. Research by Lee et al. found that polysaccharides extracted from Ginkgo Biloba episperm have inhibition effects on PEDV in vitro and have an activity superior to that of Ribavirin, as a potential anti-PEDV substance (Virus Research (20158), 195, 148-152). However, such study achievement is only limited to experiments in vitro, and the effect in vivo still needs to be proved by a large number of experiments. Chinese patent applications 2013100930843 and 201310147391.5 disclose treatment and prevention of the porcine epidemic diarrhea by using combined Chinese herbal medicine after being smashed directly. Generally, this treatment or prevention method takes a slower and limited effect, and particularly makes no effect on newborn piglets.

In a further aspect, clinically, a certain dose of a disinfectant such as povidone iodine and the like has ever been orally administered to the diseased piglets for treatment of the epidemic diarrhea, which has a certain effect and improves a survival rate by 5-10%. However, survived suckling piglets have unfavourable prognosis and grow slowly. Clinically, a supportive therapy is generally employed to help the diseased pigs to resist the disease, i.e. relief of symptoms and correction of dehydration of the diseased pigs are made by using a method of orally feeding water containing oral rehydration salts (ORS) or intraperitoneally supplementing a glucose electrolyte isotonic solution. This method achieves certain effects on weaned piglets, care pigs and adult pigs, but little effect on low-day-aged suckling piglets. In US patent application US2014/0287065A1, water containing electrolytes such as sodium hypochlorite, sodium hydroxide and the like is orally administered to pigs to reduce the probability of dehydration among PEDV-infected grown-up pigs, but the application did not provide data convincing the effect on the low-day-aged suckling piglets.

### SUMMARY OF THE INVENTION

The present invention provides a pharmaceutical composition for prevention and/or treatment of diseases induced by viruses of genus coronavirus and/or genus rotavirus to overcome the deficiencies in the prior art with lack of drugs which are available for effective treatment of porcine viral diarrhea.

In order to achieve the above objective, the present invention is realized by the following technical solution.

A pharmaceutical composition for prevention and/or treatment of diseases induced by viruses of the genus coronavirus and/or the genus rotavirus, comprises following components by weight percent: 0.5-100% of taurine, 0-85% of a vitamin complex, and 0-15% of a flavoring agent.

Taurine, also referred to as β-aminoethanesulfonic acid, was originally isolated from calculus bovis and named by this. Pure taurine is a colorless or white oblique crystal, and odorless. Taurine has a stable chemical property and is a sulfur-containing non-protein amino acid, which is existent in vivo in a free state and not engaged in biosynthesis of protein in vivo. As disclosed in the prior arts, taurine can be used for a dietary supplement or a drug. Particularly, uses of taurine as a drug are as follows: preventing and treating cardiovascular diseases, improving cardiac function and antiarrhythmia, lowering blood fat, lowering cholesterol, lowering blood pressure, lowering blood sugar, strengthening liver and benefiting gallbladder, and other effects such as antipyretic, analgesic, anti-inflammation and the like. In addition, it has been reported formerly that a mixture of taurine, anthocyanidin and Aspirin can be used for the treatment of the porcine reproductive and respiratory syndrome; and astragalus together with taurine can reduce a death rate of BALB/c mice with myocarditis induced by infection of Coxsackievirus B3. However, the prior arts do not disclose that taurine alone can be used for the treatment of the porcine viral diarrhea.

The present invention identifies through research that taurine may be used for prevention and/or treatment of a series of diseases induced by viruses of genus coronavirus, for example, porcine epidemic diarrhea and porcine transmissible gastroenteritis, and diseases such as rotavirus diarrhea and the like induced by viruses of genus rotavirus. Therefore, on the basis of above, the present invention provides a pharmaceutical composition containing taurine. The pharmaceutical composition is made by a combination of taurine, vitamin complex and flavoring agent together to realize an objective of effective treatment of diseases by taurine, by means of increasing intake of nutrient substance for animals and enhancing self-resistance of animals.

Preferably, the vitamin complex can be partially replaced by filler, and the filler is one or more of starch, sucrose, glucose and lactose. The objective of the replacement is mainly to control an intake of vitamins, such that the intake of vitamins satisfies acceptability of physiology. Definition above of the filler is only for defining the preferred implementations, and is not intended to limit the protection scope of the present invention. In fact, other types of fillers, for example, activated carbon powder, montmorillonite powder, calcium carbonate and the like, are also suitable for the present invention.

Preferably, a pharmaceutical composition for prevention and/or treatment of diseases induced by viruses of the genus coronavirus and/or the genus rotavirus, comprises the following components by weight percent: 5-100% of taurine, 0-85% of the vitamin complex, and 0-10% of the flavoring agent.

More preferably, a pharmaceutical composition for prevention and/or treatment of diseases induced by viruses of the genus coronavirus and/or the genus rotavirus, comprises the following components by weight percent: 60-90% of taurine, 5-10% of the vitamin complex, and 1-5% of the flavoring agent.

More preferably, a pharmaceutical composition for prevention and/or treatment of diseases induced by viruses of the genus coronavirus and/or the genus rotavirus, comprises the following components by weight percent: 85% of taurine, 10% of the vitamin complex, and 5% of the flavoring agent.

Preferably, the disease induced by viruses of the genus coronavirus and/or the genus rotavirus is the porcine viral diarrhea.

The porcine viral diarrhea mainly includes porcine transmissible gastroenteritis (TGE), porcine epidemic diarrhea (PED) and porcine rotavirus (PoRV). In addition, other viruses such as enterovirus infection, porcine adenovirus infection, astrovirus, calicivirus, Norwalk virus, parvovirus, pseudorabies virus and swine fever virus may also cause diarrhea of pigs.

More preferably, the porcine viral diarrhea is the diarrhea induced by a porcine epidemic diarrhea virus, a porcine transmissible gastroenteritis virus and/or a porcine rotavirus.

Preferably, the vitamin complex is one or more of vitamin A, vitamin B family, vitamin C, vitamin D₃, vitamin E, vitamin K₃, biotin, niacin, niacinamide, folic acid and inose.

Preferably, the flavoring agent is one or more of an edible aroma agent, a sweetener and a spicy flavoring agent.

More preferably, the edible aroma agent is one or more of citrus essence, strawberry essence, cream essence, vanillin and ethyl vanillin; the sweetener is one or more of sodium saccharin, xylitol, aspartame, sucralose, acesulfame potassium, glucose, sucrose and fructose; and the spicy flavoring agent is one or more of chili powder, pepper, Chinese prickly ash powder, perilla seed powder, mint powder and evodia powder. Definition above of the edible aroma agent, the sweetener and the spicy flavoring agent is only for defining the preferred implementations, and is not intended to limit the protection scope of the present invention. In fact, all types of the edible aroma agents, sweeteners and spicy flavoring agents are suitable for the present invention.

The pharmaceutical composition according to the present invention may be compounded with a corresponding adjuvant agent to prepare any clinically-acceptable dosage forms, for example, powder, granule, tablet, effervescent agent, solution, suspension, emulsion, paste, gel or the like.

As compared with the prior art, the present invention achieves the following beneficial effects:

The present invention first identifies through research that taurine can be used for prevention and/or treatment of a series of diseases induced by viruses of the genus coronavirus and/or the genus rotavirus, for example, porcine epidemic diarrhea, porcine transmissible gastroenteritis and rotavirus diarrhea which is induced by viruses of the genus rotavirus, and the like. In addition, the treatment effect of taurine on the porcine viral diarrhea is obviously better than the pharmaceutical treatment that is commonly used in the market. For better promotion of the treatment effect of taurine on the diseases, the present invention develops a pharmaceutical composition containing taurine. The pharmaceutical composition is made by the combination of taurine, the vitamin complex and the flavoring agent together to realize the objective of effective prevention or treatment of diseases by taurine, by means of increasing the intake of nutrient substance for animals and enhancing the self-resistance of animals. Because of the improvement of resistance of target animals, the administration dosage for treatment of the diseases is reduced, and the treatment effect on the diseases is improved, which may be owing to a complex synergistic effect among taurine, the vitamin complex and the flavoring agent.

### DETAILED DESCRIPTION OF THE PREFERRED EMBODIMENT

The present invention will be further described in detail by specific embodiments. Unless otherwise specified, experiment methods hereinafter are all conventional methods in the art. Unless otherwise specified, the used ingredients or materials are all ingredients or materials that are commercially available. The described below are preferred implementations of the present invention. It should be noted that for those ordinarily skilled in the art, some improvements may be made without departing from the principles of the present invention and such improvements shall be deemed as falling within the protection scope of the present invention.

### Embodiment 1 Use of Taurine in Prevention of Porcine Epidemic Diarrhea of Newborn Piglets

Prevention of the porcine epidemic diarrhea on the newborn piglets with taurine was implemented by orally administering taurine to pregnant sows.

Drug Administration Time:

3-30 days before birth of piglets until 15 days after birth of piglets. In consideration of a use-cost, the drug administration time is preferably 5-20 days before birth of piglets until 10 days after birth of piglets, and more preferably 7-14 days before birth of piglets until 5 days after birth of piglets. The effect is subject to an obvious time-effect relationship. The drug administration time for before birth of piglets directly determines a disease incidence of the newborn piglets.

Drug Administration Dosage:

each pregnant sow was administered with 5-500 g of taurine per day, and the administration was in a single dose or in divided doses. The effect is subject to an obvious quantity-effect relationship.

Specific Operations:

pregnant sows were selected and excrement thereof was detected by RT-PCR to determine the pregnant sows carrying PEDV in vivo. 60 PEDV-carried pregnant sows were divided into two groups randomly. Control group: conventional feeding; experimental group: conventional feeding+50 g per sow per day of taurine. Prevention results of the two groups are listed in Table 1. As seen from Table 1, the disease incidence and a death rate of the experimental group which was administered with taurine are obviously decreased compared to those of the control group (P<0.01).

TABLE 1

| | | Disease | |
|---|---|---|---|
| | | incidence | Death rate of |
| | | of the new | the newborn |
| Group | Dosage of taurine | born piglets (%) | piglets (%) |
| Control group | 0 | 100% | 96.6% |
| Experimental | 50 g per sow per day | 85% | 75.1% |
| group | | | |

### Embodiment 2 Use of Taurine in Treatment of the Porcine Epidemic Diarrhea of Newborn Piglets

Treatment of the porcine epidemic diarrhea on newborn piglets with taurine was implemented by orally administering a taurine solution to diseased suckling piglets and administering taurine to lactating sows, that is, simultaneous treatment of both the suckling piglets and the lactating sows.

Drug Administration Time:

the administration by drenching/mixed-feeding was carried out on the day when the disease occurred in the suckling piglets, and a non-interrupted administration lasted for 3-10 days. Lactating sows: non-interrupted oral administration lasted for 5-10 days.

Drug Administration Dosage:

the administration by drenching or mixed-feeding was carried out on the day when the disease occurred in the suckling piglets. The administration by drenching: 2% taurine solution at a dosage of 1-5 mL per time and it was taken 2-6 times per day. The administration by mixed-feeding: 1-20 g/L of taurine solution was drunk freely. Lactating sows: each sow was administered with 5-500 g of taurine per day and the administration was in a single dose or in divided doses.

Specific Operations:

40 litters of diseased newborn suckling piglets and sows thereof were selected and randomly divided into two groups equivalently. Control group: conventional supportive therapy (fluid infusion+antibiotic therapy+atropine sulfate); experimental group: treatment with taurine. Treatment results of the two groups are listed in Table 2. As seen from Table 2, a 10-day-aged survival rate and a weaned survival rate of the piglets in the experimental group are both significantly improved compared to those of the control group (P<0.05), and prognosis of the piglets is good.

TABLE 2

| | Dosage of | Dosage of | 10-day-aged | | |
|---|---|---|---|---|---|
| | taurine for the | taurine for the | survival rate | Weaned | |
| Group | lactating sows | piglets | (%) | survival rate | Prognosis |
| Control group | 0 | 0 | 8.3 | 3.2 | Poor |
| Experimental | 100 g per sow | 0.2 g per piglet | 22.5 | 20.7 | Good |
| group | per day | per day | | | |

### Embodiment 3 Use of Taurine in Prevention of the Porcine Epidemic Diarrhea of Weaned Piglets, Care Pigs and Finishing Pigs

Prevention of the porcine epidemic diarrhea on the weaned piglets, the care pigs and the finishing pigs with taurine was implemented by orally administering taurine to a swine herd, wherein the administration may be carried out by means of feeding in water or mixed feeding.

Drug Administration Time:

a preventative administration was carried out during an epidemic period of the disease, or in autumn, winter and spring when the temperature was low and changed greatly, that is, in the season when the porcine epidemic diarrhea frequently occurs. Generally, the administration can be carried out for a long-term. In consideration of the use-cost, the drug administration time is preferably 3-30 days, and more preferably, 7-14 days. The effect is subject to an obvious time-effect relationship.

Drug Administration Dosage:

0.005-2% by weight of taurine was added in daily-drinking water for the pigs to drink freely; or taurine accounting for 0.01-5% by weight of the feed was added thereto, which is equivalent to a dosage of 50-10000 mg per kilogram of body weight per day of taurine, and the administration was in a single dose or in divided doses. The effect is subject to an obvious quantity-effect relationship.

Specific Operations:

120 healthy weaned piglets, 120 healthy care pigs and 120 healthy finishing pigs were selected and randomly divided into two groups equivalently. Control group: conventional feeding; experimental group: conventional feeding+taurine (the dosage of taurine was 200 mg per kilogram of body weight per day). The prevention results of the two groups are listed in Table 3. As seen from Table 3, the disease incidence of the experimental group is lowered compared to that of the control group (P<0.05), and the survival rate of the experimental group is higher than that of the control group (P<0.05).

TABLE 3

| | | Disease | Survival | |
|---|---|---|---|---|
| | | incidence | rate | |
| Group | | (%) | (%) | Prognosis |
| Weaned piglets | Control group | 10 | 91.6 | Normal |
| | Experimental group | 6.6 | 96.6 | Excellent |
| Care pigs | Control group | 8.3 | 95.0 | Normal |
| | Experimental group | 3.3 | 96.6 | Excellent |
| Finishing pigs | Control group | 3.3 | 96.6 | Normal |
| | Experimental group | 0 | 98.3 | Excellent |

### Embodiment 4 Use of Taurine in Treatment of the Porcine Epidemic Diarrhea of Weaned Piglets, Care Pigs and Finishing Pigs

Treatment of the porcine epidemic diarrhea on the weaned piglets, the care pigs and the finishing pigs with taurine was implemented by orally administering taurine to the pigs, wherein the administration may be carried out by means of feeding in water or mixed feeding.

Drug Administration Time:

the administration was carried out from the day of occurrence of the disease, and in consideration of the use-cost, the drug administration time is preferably 3-30 days, and more preferably 7-14 days. The effect is subject to an obvious time-effect relationship.

Drug Administration Dosage:

0.1-2% by weight of taurine was added in the daily-drinking water for the pigs to drink freely; or taurine accounting for 0.2-5% of the feed was added thereto, which is equivalent to a dosage of 100-10000 mg per kilogram of body weight per day of taurine, and the administration was in a single dose or in divided doses. The effect is subject to an obvious quantity-effect relationship.

Specific Operations:

60 diseased weaned piglets, 60 diseased care pigs and 60 diseased finishing pigs were selected, and were randomly divided into two groups equivalently. Control group: conventional supportive therapy (fluid infusion+antibiotic therapy+atropine sulfate); experimental group: treatment with taurine (the dosage of taurine was 300 mg per kilogram of body weight per day). The treatment results of the two groups are listed in Table 4. As seen from Table 4, the survival rate of the experimental group is higher than that of the control group (P<0.05).

TABLE 4

| Group | | Survival rate (%) | Prognosis |
|---|---|---|---|
| Weaned piglets | Control group | 86.6 | General |
| | Experimental group | 90.0 | Good |
| Care pigs | Control group | 90.0 | General |
| | Experimental group | 93.3 | Good |
| Finishing pigs | Control group | 96.6 | General |
| | Experimental group | 100 | Good |

### Embodiment 5

According to methods as described in Embodiment 2 and Embodiment 4, treatment effects of taurine on porcine transmissible gastroenteritis, rotavirus diarrhea and pseudorabies viral diarrhea of the newborn piglets, the weaned piglets, the care pigs and the finishing pigs were tested. The results are shown in Table 5. The dosage of taurine for the newborn piglets was the same as that in Embodiment 2; and the dosage of taurine for the weaned piglets, the care pigs and the finishing pigs was the same as that in Embodiment 4. As seen from Table 5, taurine can be used for treatment of the porcine transmissible gastroenteritis and the rotavirus diarrhea, but the treatment effects of taurine on the porcine transmissible gastroenteritis and the rotavirus diarrhea are poorer than that on the porcine epidemic diarrhea. However, taurine has no treatment effect on the pseudorabies viral diarrhea, for following reasons: in one aspect, a pseudorabies virus which belongs to a virus of genus porcine herpesvirus of family herpesviridae, is a virus having relatively strong resistibility among the family herpesviridae and still has infectivity after being treated with 0.5% phenol for 32 days; in another aspect, the category of the virus may be also a factor causing failure of the treatment effect. The porcine pseudorabies virus is a DNA virus, whereas the porcine epidemic diarrhea virus, the porcine transmissible gastroenteritis virus and the rotavirus all belong to RNA viruses.

TABLE 5

| | | | Survival rate | |
|---|---|---|---|---|
| Group | | | (%) | Prognosis |
| Transmissible | Newborn piglets | Control group | 9.8 | Poor |
| gastroenteritis | | Experimental group | 16.6 | Good |
| | Weaned piglets | Control group | 83.3 | Poor |
| | | Experimental group | 90 | Good |
| | Care pigs | Control group | 90 | Poor |
| | | Experimental group | 93.3 | Good |
| | Finishing pigs | Control group | 96.6 | Poor |
| | | Experimental group | 96.6 | Good |
| Rotavirus diarrhea | Newborn piglets | Control group | 88.7 | Poor |
| | | Experimental group | 93.6 | Good |
| | Weaned piglets | Control group | 90 | General |
| | | Experimental group | 96.6 | Good |
| | Care pigs | Control group | 93.3 | General |
| | | Experimental group | 96.6 | Good |
| Pseudorabies viral | Newborn piglets | Control group | 0 | — |
| diarrhea | | Experimental group | 0 | — |
| | Care pigs | Control group | 46.6 | General |
| | | Experimental group | 43.3 | General |

### Embodiment 6

A pharmaceutical composition comprises following components by weight percent: 85% of taurine, 10% of a vitamin complex, and 5% of a flavoring agent.

The vitamin complex consists of vitamin A, vitamin B₁, vitamin B₂, vitamin B₆, vitamin B₁₂, vitamin C, vitamin D₃, vitamin E, vitamin K₃, biotin, niacin, calcium pantothenate, folic acid and inose.

The flavoring agent consists of vanillin, aspartame and chili powder.

### Embodiment 7

A pharmaceutical composition comprises the following components by weight percent: 5% of taurine, 85% of the vitamin complex, and 10% of the flavoring agent.

The vitamin complex consists of vitamin A, vitamin B₁, vitamin B₂, vitamin B₆, vitamin B₁₂, vitamin C, 25-hydroxyl-vitamin D₃, vitamin E, vitamin K₃, niacinamide, pantothenic acid, folic acid and glucose.

The flavoring agent consists of ethyl vanillin, acesulfame potassium and perilla seed powder.

### Embodiment 8

A pharmaceutical composition comprises the following components by weight percent: 70% of taurine, 25% of the vitamin complex, and 5% of the flavoring agent.

The vitamin complex consists of vitamin A, vitamin B₁₂, vitamin C, 25-hydroxyl-vitamin D₃, vitamin E, vitamin K₃, niacinamide, pantothenic acid, folic acid, inose and lactose.

The flavoring agent consists of citrus essence, sucralose and evodia powder.

### Embodiment 9

A pharmaceutical composition comprises the following components by weight percent: 45% of taurine, 45% of the vitamin complex, and 10% of the flavoring agent.

The vitamin complex consists of vitamin A, vitamin C, vitamin D₃, vitamin E, vitamin K₃, biotin, niacinamide, pantothenic acid, folic acid, soluble starch and sucrose.

The flavoring agent consists of strawberry essence, sodium saccharin and mint powder.

### Embodiment 10

A pharmaceutical composition comprises the following components by weight percent: 20% of taurine, 70% of the vitamin complex, and 10% of the flavoring agent.

The vitamin complex consists of vitamin B₁, vitamin B₂, vitamin B₆, vitamin B₁₂, vitamin C, vitamin E, vitamin K₃, niacinamide, pantothenic acid, folic acid, starch, lactose, sucrose and glucose.

The flavoring agent consists of cream essence, xylitol, glucose and pepper.

### Embodiment 11

I. The pharmaceutical composition according to Embodiment 6 was used for the prevention of porcine epidemic diarrhea of the newborn piglets, weaned piglets, care pigs and finishing pigs. Particularly, a mode of the administration for the newborn piglets was the same as the method in Embodiment 1, i.e. each pregnant sow was administered with 50 g of the pharmaceutical composition in Embodiment 6 per day, and the administration was in a single dose or in divided doses. A mode of the administration for the weaned piglets, care pigs and finishing pigs was the same as the method in Embodiment 3, i.e. a dosage of 250 mg per kilogram of body weight per day of the pharmaceutical composition in Embodiment 6, and the administration was in a single dose or in divided doses.

The prevention results of the pharmaceutical composition according to Embodiment 6 for the porcine epidemic diarrhea of newborn piglets, weaned piglets, care pigs and finishing pigs are shown in Tables 6 and 7.

TABLE 6
Prevention results of the newborn piglets

| | Dosage of the | Disease incidence | |
|---|---|---|---|
| | pharmaceutical | of the | Death rate of |
| | composition | newborn | the newborn |
| Group | for the sows | piglets (%) | piglets (%) |
| Control group | 0 | 100% | 100% |
| Experimental | 50 g per sow per day | 83% | 73.9% |
| group | | | |

TABLE 7
Prevention results of the weaned piglets, care pigs and finishing pigs

| | | Disease | | |
|---|---|---|---|---|
| | | incidence | Survival | |
| Group | | (%) | rate (%) | Prognosis |
| Weaned piglets | Control group | 13.3 | 88.3 | Normal |
| | Experimental group | 8.3 | 91.6 | Excellent |
| Care pigs | Control group | 11.6 | 93.3 | Normal |
| | Experimental group | 6.6 | 96.6 | Excellent |
| Finishing pigs | Control group | 3.3 | 96.6 | Normal |
| | Experimental group | 0 | 98.3 | Excellent |

II. The pharmaceutical composition according to Embodiment 6 was used for the treatment of porcine epidemic diarrhea of the newborn piglets, weaned piglets, care pigs and finishing pigs. A mode of the administration for the newborn piglets was the same as the method in Embodiment 2, i.e. the administration by drenching and/or mixed-feeding was carried out on the day when the disease occurred in the suckling piglets. The administration by drenching: a solution with a mass concentration of 5% was prepared from the pharmaceutical composition in Embodiment 6 with a dosage of 2-4 mL per time and it was taken 2-6 times per day. The administration by mixed-feeding: 4 g/L of the solution was drunk freely. For lactating sows: each sow was administered with 100 g of the pharmaceutical composition in Embodiment 6 per day and the administration was in a single dose or in divided doses. A mode of the administration for the weaned piglets, care pigs and finishing pigs was the same as the method in Embodiment 4, i.e. a dosage of 500 mg per kilogram of body weight per day of the pharmaceutical composition in Embodiment 6, and the administration was in a single dose or in divided doses.

The treatment results of the pharmaceutical composition according to Embodiment 6 for the porcine epidemic diarrhea of newborn piglets, weaned piglets, care pigs and finishing pigs are shown in Tables 8 and 9.

TABLE 8
Treatment results of the newborn piglets

| | Dosage of the | Dosage of the | | | |
|---|---|---|---|---|---|
| | composition for | pharmaceutical | 10-day-aged | Weaned | |
| | the lactating | composition for | survival rate | survival rate | |
| Group | sows | the piglets | (%) | (%) | Prognosis |
| Control group | 0 | 0 | 7.7 | 2.9 | Poor |
| Experimental | 100 g per sow | 0.2 g per piglet | 23.3 | 22.6 | Good |
| group | per day | per day | | | |

TABLE 9
Treatment results of the weaned piglets, care pigs and finishing pigs

| Group | | Survival rate (%) | Prognosis |
|---|---|---|---|
| Weaned piglets | Control group | 86.6 | General |
| | Experimental group | 93.3 | Excellent |
| Care pigs | Control group | 90.0 | General |
| | Experimental group | 96.6 | Excellent |
| Finishing pigs | Control group | 96.6 | General |
| | Experimental group | 100 | Excellent |

III. The treatment effects of the pharmaceutical composition in Embodiment 6 on the porcine transmissible gastroenteritis and the porcine rotavirus diarrhea of the newborn piglets, weaned piglets, care pigs and finishing pigs were tested according to the method in the second step in the present embodiment. A dosage of the pharmaceutical composition for the newborn piglets was: 120 g per lactating sow per day, and 0.2 g per piglet per day. A dosage of the pharmaceutical composition for the weaned piglets, care pigs and finishing pigs was 350 mg per kilogram of body weight. The results are shown in Table 10.

TABLE 10

| | | | Survival rate | |
|---|---|---|---|---|
| Group | | | (%) | Prognosis |
| Transmissible | Newborn piglets | Control group | 11.8 | Poor |
| gastroenteritis | | Experimental group | 18.2 | Excellent |
| | Weaned piglets | Control group | 83.3 | Poor |
| | | Experimental group | 86.6 | Excellent |
| | Care pigs | Control group | 90 | Poor |
| | | Experimental group | 93.3 | Excellent |
| | Finishing pigs | Control group | 93.3 | Poor |
| | | Experimental group | 96.6 | Excellent |
| Rotavirus diarrhea | Newborn piglets | Control group | 90.5 | Poor |
| | | Experimental group | 93.6 | Excellent |
| | Weaned piglets | Control group | 90 | General |
| | | Experimental group | 93.3 | Excellent |
| | Care pigs | Control group | 93.3 | General |
| | | Experimental group | 96.6 | Excellent |

### Embodiment 12

I. The pharmaceutical composition in Embodiment 7 was used for the prevention of porcine epidemic diarrhea of the newborn piglets, weaned piglets, care pigs and finishing pigs. An administration method was the same as the method in Embodiment 11, and 2000 mg per kilogram of body weight of the pharmaceutical composition in Embodiment 7 was administered to the weaned piglets, care pigs and finishing pigs. The prevention results of the composition according to Embodiment 7 for the porcine epidemic diarrhea of newborn piglets, weaned piglets, care pigs and finishing pigs are shown in Tables 11 and 12.

TABLE 11
Prevention results of the newborn piglets

| | Dosage of the | | |
|---|---|---|---|
| | pharmaceutical | Disease incidence of the | Death rate of the newborn |
| Group | composition for the sows | newborn piglets (%) | piglets (%) |
| Control group | 0 | 100% | 98.3% |
| Experimental group | 400 g per sow per day | 92.2% | 85.1% |

TABLE 12
Prevention results of the weaned piglets, care pigs and grow-finishing pigs

| | | Disease | | |
|---|---|---|---|---|
| | | incidence | Survival | |
| Group | | (%) | rate (%) | Prognosis |
| Weaned piglets | Control group | 10.0 | 88.3 | Normal |
| | Experimental group | 8.3 | 91.6 | Excellent |
| Care pigs | Control group | 8.3 | 93.3 | Normal |
| | Experimental group | 6.6 | 96.6 | Excellent |
| Finishing pigs | Control group | 3.3 | 96.6 | Normal |
| | Experimental group | 0 | 98.3 | Excellent |

II. The pharmaceutical composition in Embodiment 7 was used for the treatment of porcine epidemic diarrhea of the newborn piglets, weaned piglets, care pigs and finishing pigs. The administration method was the same as the method in Embodiment 11, and 5000 mg per kilogram of body weight of the pharmaceutical composition in Embodiment 7 was administered to the weaned piglets, care pigs and finishing pigs. The treatment results of the composition according to Embodiment 7 for the porcine epidemic diarrhea of newborn piglets, weaned piglets, care pigs and finishing pigs are shown in Tables 13 and 14.

TABLE 13
Treatment results of the newborn piglets

| | Dosage of the | | | | |
|---|---|---|---|---|---|
| | composition for | Dosage of the | 10-day-aged | Weaned | |
| | the lactating | composition for | survival rate | survival rate | |
| Group | sows | the piglets | (%) | (%) | Prognosis |
| Control group | 0 | 0 | 8.9 | 3.3 | Poor |
| Experimental | 400 g per sow | 1 g per piglet per | 12.7 | 12.3 | Excellent |
| group | per day | day | | | |

TABLE 14
Treatment results of the weaned piglets, care pigs and finishing pigs

| Group | | Survival rate (%) | Prognosis |
|---|---|---|---|
| Weaned piglets | Control group | 90.0 | General |
| | Experimental group | 93.3 | Excellent |
| Care pigs | Control group | 93.3 | General |
| | Experimental group | 96.6 | Excellent |
| Finishing pigs | Control group | 96.6 | General |
| | Experimental group | 100 | Excellent |

III. The treatment effects of the pharmaceutical composition in Embodiment 7 on the porcine transmissible gastroenteritis and the porcine rotavirus diarrhea of the newborn piglets, weaned piglets, care pigs and finishing pigs were tested according to the steps in Embodiment 11. A dosage of the pharmaceutical composition for the newborn piglets was: 2000 g per lactating sow per day, and 4 g per piglet per day. A dosage of the composition for the weaned piglets, care pigs and finishing pigs was 5000 mg per kilogram of body weight. The results are shown in Table 15.

TABLE 15

| | | | Survival rate | |
|---|---|---|---|---|
| Group | | | (%) | Prognosis |
| Transmissible | Newborn piglets | Control group | 6.8 | Poor |
| gastroenteritis | | Experimental group | 13.2 | Excellent |
| | Weaned piglets | Control group | 83.3 | Poor |
| | | Experimental group | 90.0 | Excellent |
| | Care pigs | Control group | 86.6 | Poor |
| | | Experimental group | 93.3 | Excellent |
| | Finishing pigs | Control group | 96.6 | Poor |
| | | Experimental group | 96.6 | Excellent |
| Rotavirus diarrhea | Newborn piglets | Control group | 91.2 | Poor |
| | | Experimental group | 92.8 | Excellent |
| | Weaned piglets | Control group | 90 | General |
| | | Experimental group | 93.3 | Excellent |
| | Care pigs | Control group | 86.6 | General |
| | | Experimental group | 93.3 | Excellent |

### Embodiment 13

I. The pharmaceutical composition in Embodiment 9 was used for the prevention of porcine epidemic diarrhea of the newborn piglets, weaned piglets, care pigs and finishing pigs. The administration method was the same as the method in Embodiment 11, and 450 mg per kilogram of body weight of the pharmaceutical composition in Embodiment 9 was administered to the weaned piglets, care pigs and finishing pigs. The prevention results of the composition in Embodiment 9 on the porcine epidemic diarrhea of newborn piglets, weaned piglets, care pigs and finishing pigs are shown in Tables 16 and 17.

TABLE 16
Prevention results of the newborn piglets

| | Dosage of the | | |
|---|---|---|---|
| | pharmaceutical | | |
| | composition for the | Disease incidence of the | Death rate of the newborn |
| Group | sows | newborn piglets (%) | piglets (%) |
| Control group | 0 | 90.6% | 96.9% |
| Experimental | 300 g per sow per day | 70.9% | 71.2% |
| group | | | |

TABLE 17
Prevention results of the weaned piglets, care pigs and finishing pigs

| | | Disease | | |
|---|---|---|---|---|
| | | incidence | Survival | |
| Group | | (%) | rate (%) | Prognosis |
| Weaned piglets | Control group | 11.6 | 88.3 | General |
| | Experimental group | 8.3 | 93.3 | Excellent |
| Care pigs | Control group | 10.0 | 93.3 | General |
| | Experimental group | 6.6 | 96.6 | Excellent |
| Finishing pigs | Control group | 3.3 | 96.6 | Normal |
| | Experimental group | 0 | 98.3 | Excellent |

II. The pharmaceutical composition in Embodiment 9 was used for the treatment of porcine epidemic diarrhea of the newborn piglets, weaned piglets, care pigs and finishing pigs. The administration method was the same as the method in Embodiment 11, and 800 mg per kilogram of body weight of the pharmaceutical composition in Embodiment 9 was administered to the weaned piglets, care pigs and finishing pigs. The treatment results of the composition in Embodiment 9 for the porcine epidemic diarrhea of the newborn piglets, weaned piglets, care pigs and finishing pigs are as shown in Tables 18 and 19.

TABLE 18
Treatment results of the newborn piglets

| | Dosage of the | | | | |
|---|---|---|---|---|---|
| | composition for | Dosage of the | 10-day-aged | Weaned | |
| | the lactating | composition for | survival rate | survival rate | |
| Group | sows | the piglets | (%) | (%) | Prognosis |
| Control group | 0 | 0 | 6.3 | 4.5 | Poor |
| Experimental | 500 g per sow | 0.8 g per piglet | 27.7 | 26.3 | Excellent |
| group | per day | per day | | | |

TABLE 19
Treatment results of the weaned piglets, care pigs and finishing pigs

| Group | | Survival rate (%) | Prognosis |
|---|---|---|---|
| Weaned piglets | Control group | 90.0 | General |
| | Experimental group | 96.6 | Excellent |
| Care pigs | Control group | 93.3 | General |
| | Experimental group | 96.6 | Excellent |
| Finishing pigs | Control group | 96.6 | General |
| | Experimental group | 100 | Excellent |

III. The treatment effects of the pharmaceutical composition in Embodiment 9 on the porcine transmissible gastroenteritis and the rotavirus diarrhea of the newborn piglets, weaned piglets, care pigs and finishing pigs were tested according to the steps in Embodiment 11. A dosage of the composition for the newborn piglets was: 200 g per lactating sow per day, and 0.4 g per piglet per day. A dosage of the composition for the weaned piglets, care pigs and finishing pigs was 500 mg per kilogram of body weight. The results are shown in Table 20.

TABLE 20

| | | | Survival rate | |
|---|---|---|---|---|
| Group | | | (%) | Prognosis |
| Transmissible | Newborn piglets | Control group | 10.5 | Poor |
| gastroenteritis | | Experimental group | 14.8 | Excellent |
| | Weaned piglets | Control group | 83.3 | Poor |
| | | Experimental group | 93.3 | Excellent |
| | Care pigs | Control group | 86.6 | Poor |
| | | Experimental group | 93.3 | Excellent |
| | Finishing pigs | Control group | 96.6 | Poor |
| | | Experimental group | 100 | Excellent |
| Rotavirus diarrhea | Newborn piglets | Control group | 90.3 | Poor |
| | | Experimental group | 93.9 | Excellent |
| | Weaned piglets | Control group | 93.3 | General |
| | | Experimental group | 93.3 | Excellent |
| | Care pigs | Control group | 86.6 | General |
| | | Experimental group | 93.3 | Excellent |

1. A pharmaceutical composition for prevention and/or treatment of diseases induced by viruses of genus coronavirus and/or genus rotavirus, characterized in that, it comprises following components by weight percent: 0.5-100% of taurine, 0-85% of a vitamin complex, and 0-15% of a flavoring agent.

2. The pharmaceutical composition according to claim 1, wherein it comprises the following components by weight percent: 5-100% of taurine, 0-85% of the vitamin complex, and 0-10% of the flavoring agent.

3. The pharmaceutical composition according to claim 1, wherein it comprises the following components by weight percent: 60-90% of taurine, 5-10% of the vitamin complex, and 1-5% of the flavoring agent.

4. The pharmaceutical composition according to claim 1, wherein it comprises the following components by weight percent: 85% of taurine, 10% of the vitamin complex, and 5% of the flavoring agent.

5. The pharmaceutical composition according to claim 1, wherein the disease induced by viruses of the genus coronavirus and/or the genus rotavirus is porcine viral diarrhea.

6. The pharmaceutical composition according to claim 5, wherein the porcine viral diarrhea is the diarrhea induced by a porcine epidemic diarrhea virus, a porcine transmissible gastroenteritis virus and/or a porcine rotavirus.

7. The pharmaceutical composition according to claim 1, wherein the vitamin complex is partially replaceable by a filler, the filler being one or more of starch, sucrose, glucose and lactose.

8. The pharmaceutical composition according to claim 1, wherein the vitamin complex is one or more of vitamin A, vitamin B family, vitamin C, vitamin D₃, 25-hydroxyl-vitamin D₃, vitamin E, vitamin K₃, biotin, niacin, niacinamide, pantothenic acid, calcium pantothenate, folic acid and inose.

9. The pharmaceutical composition according to claim 14, wherein the flavoring agent is one or more of an edible aroma agent, a sweetener and a spicy flavoring agent.

10. The pharmaceutical composition according to claim 1, wherein the pharmaceutical composition is compounded with a corresponding adjuvant agent to prepare a clinically-acceptable dosage form.

11. The pharmaceutical composition according to claim 2, wherein the disease induced by viruses of the genus coronavirus and/or the genus rotavirus is the porcine viral diarrhea.

12. The pharmaceutical composition according to claim 3, wherein the disease induced by viruses of the genus coronavirus and/or the genus rotavirus is the porcine viral diarrhea.

13. The pharmaceutical composition according to claim 4, wherein the disease induced by viruses of the genus coronavirus and/or the genus rotavirus is the porcine viral diarrhea.

14. The pharmaceutical composition according to claim 11, wherein the porcine viral diarrhea is the diarrhea induced by the porcine epidemic diarrhea virus, the porcine transmissible gastroenteritis virus and/or the porcine rotavirus.

15. The pharmaceutical composition according to claim 12, wherein the porcine viral diarrhea is the diarrhea induced by the porcine epidemic diarrhea virus, the porcine transmissible gastroenteritis virus and/or the porcine rotavirus.

16. The pharmaceutical composition according to claim 13, wherein the porcine viral diarrhea is the diarrhea induced by the porcine epidemic diarrhea virus, the porcine transmissible gastroenteritis virus and/or the porcine rotavirus.

17. The pharmaceutical composition according to claim 2, wherein the pharmaceutical composition is compounded with the corresponding adjuvant agent to prepare the clinically-acceptable dosage form.

18. The pharmaceutical composition according to claim 3, wherein the pharmaceutical composition is compounded with the corresponding adjuvant agent to prepare the clinically-acceptable dosage form.

19. The pharmaceutical composition according to claim 4, wherein the pharmaceutical composition is compounded with the corresponding adjuvant agent to prepare the clinically-acceptable dosage form.
